(19) United States
(12) Patent Application Publication
(10) Pub. No.: US 2025/0230384 A1
(43) Pub. Date: Jul. 17, 2025

(54) HAND DISH-WASHING COMPOSITION CONTAINING ETHOXYLATED GLYCEROL ESTERS

(71) Applicant: CLARIANT INTERNATIONAL LTD, Muttenz (CH)

(72) Inventor: Natascha SCHELERO, Hattersheim (DE)

(21) Appl. No.: 18/703,756

(22) PCT Filed: Nov. 10, 2022
(86) PCT No.: PCT/EP2022/081543

§ 371 Date: Apr. 23, 2024

(30) Foreign Application Priority Data
Nov. 12, 2021 (EP) 21207986.7

(51) Int. Cl. C11D 1/74; B01J 23/02; C07C 41/16; C11D 3/00

(52) U.S. Cl. CPC C11D 1/74; C07C 41/16; C11D 3/0026; B01J 23/02; C11D 2111/14

(57) ABSTRACT

A hand dish-washing composition which comprises one or more ethoxylated glycerol esters prepared in the presence of a calcium-based catalyst, which is useful for providing anti-foaming activity upon rinse and for providing a water-sheeting effect resulting in faster and spotless drying.

The present invention relates to a hand dish-washing composition, comprising one or more ethoxylated glycerol esters prepared in the presence of a calcium-based catalyst, said composition maintaining initial foam and cleaning performance while showing significant foam reduction during rinse.

Available and usable water resources on earth are getting less and less. It is estimated that by 2025 two-third of world's population will lack access to sufficient water resources. To combat the threat of water scarcity, water consumption needs to be reduced. This can be realized by reducing the amount of water being used or by reusing water.

Necessary household routines such as laundry, dishwashing, and other cleaning processes, consume large amounts of water around the world. Water and detergent consumption for these routines cannot be avoided. Foaming of liquid detergent in hand dish washing application is associate with its efficiency by consumers. However, rinsing off the foam remaining on the cleaned dishes, consumes additional water.

The amount of water required for rinse depends on the composition of detergent used. Therefore, optimizing the composition of a detergent can be used to minimize water consumption during rinse.

As a result, there is a need for new cleaning ingredients that can be successfully employed in the field of cleaning compositions, in particular hand dish-washing compositions, having foaming and cleaning characteristics during wash, and in addition exhibits an antifoaming effect during rinse.

U.S. Pat. No. 4,904,359 discloses a detergent composition containing anionic surfactant and polymeric surfactant (Pluronic Polyethylendiamine) which form stable surfactant-polymer-complexes. The disclosed detergent composition is characterized by its improved grease removal and high sud. Any impact on antifoaming effect during rise is not described.

WO95/23204 discloses a high foaming liquid detergent with desirable cleansing properties and mildness to human skin, comprising 1-23% of partially esterified ethoxylated polyhydric alcohol, 0-30% nonionic surfactants which are primary and secondary C8-18 alkanol and/or alkylphenol condensates with 50-30 moles ethylenoxide, 0-40% water dispersible anionic surfactants and 0-10% betaine. There is no disclosure that the high foam volume comes across with easy rinse benefit.

WO2014/138327 discloses a good foaming detergent while less surfactant content-containing a nonionic, amphiphilic polymer and surfactants. The polymer is a copolymer of acrylate und polyvinylpyrrolidone. Although the detergent contains less surfactant, no impact on an antifoaming effect during rinse is disclosed.

WO2017/058934 disclose a detergent formulation generating stable flash foam. The detergent formulation consists of betaine, and a linear alcohol ethoxylate, semipolar nonionic surfactant. The linear alcohol ethoxylate comprises a fatty alcohol with 6-18 carbons.

WO2016/030226 A1 describes a detergent formulation containing in addition to the standard surfactant mixture a nonionic surfactant type of Laureth 5, 7, 9 and mixture of lauric and steric fatty acid which has the benefit of stable foam during wash but easy to rinse.

It is an object of the invention to provide a new cleaning ingredient for hand dish-washing compositions that helps to use less water during rinsing in manual dish cleaning.

It is another object of the present invention to provide a new cleaning ingredient for hand dish-washing composition that has an antifoaming effect during rinse while maintaining foaming characteristics during wash.

Surprisingly, it has been found that hand dish-washing compositions based on one or more ethoxylated glycerol esters prepared in the presence of a calcium-based catalyst show a water-sheeting effect, and provide an antifoaming effect only during rinse in manual dish-washing.

WO97/18284 describes a detergent formulation that contains solvent hydrotrope from the group consisting of alkoxylated glycerides, alkoxylated glycerines, alkoxylated fatty acids, esters of glycerin, polyglycerol esters and combination thereof (1-30 wt %). There is no disclosure that the formulation contains ethoxylated glycerol esters prepared in the presence of a calcium catalyst, and that the composition has an antifoaming effect during rinse in addition to its standard foaming behavior.

EP 2 666 848 A1 discloses a concentrated dilutable cleaning composition comprising a mixture of polyethoxylated glycerin ester compounds with one, two or three ester residues. The disclosed mixtures contain a major amount of glycerol monoesters. They are synthesized in the presence of potassium hydroxide as catalyst. They are proposed for use in manual dish-washing compositions, however, there is no disclosure that the composition has an antifoaming effect during rinse in addition to its standard foaming behavior.

WO 2020/239750 A1 and WO 2020/239760 A1 disclose ethoxylated glycerol esters obtained in the presence of specific calcium catalysts, however, a use of such compounds in manual dish-washing compositions is not contemplated in these documents.

Accordingly, in one aspect of the invention there is provided a hand dish-washing composition which comprises
Z1) a component comprising one or more ethoxylated glycerol esters of the formula (I)

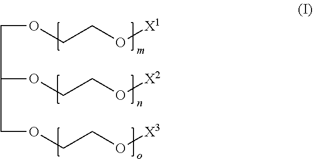

wherein
X¹ is —C(═O)—R¹ or H, X² is —C(═O)—R² or H, X³ is —C(═O)—R³ or H with the proviso that not all of the groups X¹, X² and X³ are H;
R¹, R² and R³ are equal or different and are independently selected from saturated or unsaturated, linear or branched C₇-C₂₄ aliphatic groups; and
m, n and o are equal or different and are each independently an integer number from 1 to 200, preferably from 1 to 80, more preferably from 2 to 70, with the
from the preparation of one proviso that the number-average of the sum of m+n+o is greater than 2, preferably greater than 5, more preferably from 20 to 70, even more preferably from 30 to 60;
wherein component Z1) is a product mixture or more ethoxylated glycerol esters of the formula (I) in the presence of a calcium-based catalyst, preferably a product mixture from the preparation of one or more ethoxylated glycerol esters of the formula (I) by ethoxylating one or more triglycerides in the presence of a calcium-based catalyst;
Z2) a surfactant component, comprising one or more anionic surfactants;
Z3) a pH adjustment component, adjusting the pH value of the composition to from 4.5 to 8.5;
Z4) optionally further ingredients, and
Z5) a solvent component.

In a further aspect of the invention there is provided a process of reducing foam during rinse in hand dish-washing comprising the steps of:
a. applying onto the hard surface in neat or diluted form a liquid hand dish-washing composition according to the invention;
b. cleaning the hard surface with a cleaning implement; and
c. rinsing the hard surface.

In yet a further aspect of the invention there is provided the use of a hand dish-washing composition according to the invention for providing anti-foaming activity upon rinse.

In yet a further aspect of the invention there is provided the use of a hand dish-washing composition according to the invention for providing a water-sheeting effect resulting in faster and spotless drying.

In yet a further aspect of the invention there is provided the use of the component Z1 according to the invention in a hand dish-washing composition.

In yet a further aspect of the invention there is provided the use of the component Z1 according to the invention in a hand dish-washing composition for providing a water-sheeting effect resulting in faster and spotless drying.

In the context of the present invention, the “water-sheeting effect” means the spread-out of water over a surface forming a sheet that slides away from the surface. This results in faster and spotless drying of the surface.

A subject matter of the invention is the use of a hand dish-washing composition according to the invention for providing faster and spotless drying, in particular of dishware.

A subject matter of the invention is the use of component Z1 according to the invention in a hand dish-washing composition for providing faster and spotless drying, in particular of dishware.

### Ethoxylated Glycerol Ester Component Z1)

The hand dish-washing composition of the invention comprises a component Z1) comprising one or more ethoxylated glycerol esters of the formula (I)

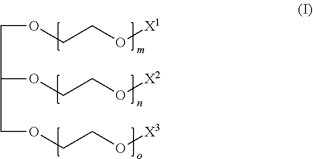

wherein
X¹ is —C(═O)—R¹ or H, X² is —C(═O)—R² or H, X³ is —C(═O)—R³ or H with the proviso that not all of the groups X¹, X² and X³ are H;
R¹, R² and R³ are equal or different and are independently selected from saturated or unsaturated, linear or branched C₇-C₂₄ aliphatic groups; and
m, n and o are equal or different and are each independently an integer number from 1 to 200, preferably from 1 to 80, more preferably from 2 to 70, with the proviso that the number-average of the sum of m+n+o is greater than 2, preferably greater than 5, more preferably from 20 to 70, even more preferably from 30 to 60;
wherein component Z1) is a product mixture from the preparation of one or more ethoxylated glycerol esters of the formula (I) in the presence of a calcium-based catalyst.

In a preferred embodiment, component Z1) of the hand dish-washing composition according to the invention further comprises, in addition to the one or more ethoxylated glycerol esters of the formula (I), one or more polyethyleneglycol diesters of the formula (XV)

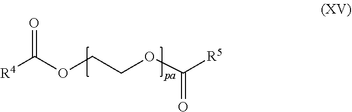

wherein
R⁴ and R⁵ are equal or different and are independently selected from saturated or unsaturated, linear or branched C₇-C₂₄ aliphatic groups; and
pa is an integer number from 1 to 200, preferably from 1 to 80, more preferably from 5 to 30 and even more preferably from 10 to 20.

Component Z1) of the hand dish-washing composition according to the invention may comprise, in addition to the one or more ethoxylated glycerol esters of the formula (I) and optionally the one or more polyethyleneglycol diesters of the formula (XV), one or more further substances selected from the group consisting of polyethyleneglycol monoesters of the formula (XIX)

R⁶—COO—[CH₂CH₂O]_{pw}—H (XIX)
wherein
R⁶ is selected from saturated or unsaturated, linear or branched C₇-C₂₄ aliphatic groups; and
pw is an integer number from 1 to 200, preferably from 1 to 80, more preferably from 5 to 30 and even more preferably from 10 to 20; and
polyethyleneglycols of the formula (XX)

HO—[CH₂CH₂O]ₚₓ—H (XX)
wherein
px is an integer number from 1 to 200, preferably from 1 to 80, more preferably from 5 to 80 and even more preferably from 10 to 80.

Component Z1) of the hand dish-washing composition according to the invention is a product mixture from the preparation of one or more ethoxylated glycerol esters of the formula (I) in the presence of a calcium-based catalyst, preferably a product mixture from the preparation of one or more ethoxylated glycerol esters of the formula (I) by ethoxylating one or more triglycerides, preferably one or more triglycerides of the formula (II), in the presence of a calcium-based catalyst, preferably of a calcium-based catalyst as described below.

In a preferred embodiment of the invention, component Z1) of the hand dish-washing composition according to the invention is prepared from 1 to 200 mol ethylene oxide, preferably from 1 to 80 mol ethylene oxide, more preferably from 20 to 70 mol ethylene oxide and even more preferably from 30 to 60 mol ethylene oxide; and 1 mol of one or more triglycerides of the formula (II)

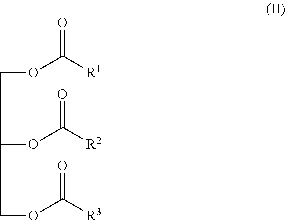

in the presence of a calcium-based catalyst,
wherein R¹, R² and R³ in formula (II) are equal or different and are independently selected from saturated or unsaturated, linear or branched C₇-C₂₄ aliphatic groups.

Preferably, component Z1) is prepared with a calcium-based catalyst (C) obtainable by a reaction involving
(A) calcium hydroxide and
(B) a carboxylic acid comprising 3 to 40 carbon atoms,
wherein the molar ratio of calcium hydroxide (A) to carboxylic acid (B) in the preparation of the catalyst (C) is from 1:1 to 1:5. More preferably, the molar ratio (A):(B) is from 1:1.5 to 1:4, even more preferably from 1:1.8 to 1:2.2, and particularly preferably from 1:1.9 to 1:2.1.

The reaction for the preparation of the catalyst (C) involving calcium hydroxide (A) and carboxylic acid (B) is preferably carried out in the presence of at least one polar solvent, more preferably at least one polar solvent comprising at least one hydroxyl group and even more preferably at least one alcohol having 1 to 5 carbon atoms or a mixture thereof with water. In a particularly preferred embodiment, the polar solvent is propan-2-ol or a mixture thereof with water.

Preferably, the reaction for the preparation of the catalyst (C) involves a carboxylic acid (B) represented by formula (III) or formula (IV)

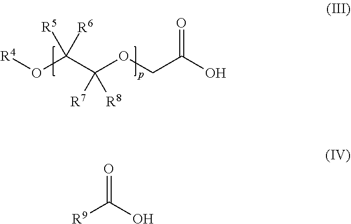

wherein
R⁴ in formula (III) is selected from saturated or unsaturated, linear or branched C₁-C₃₀ aliphatic groups, preferably C₅-C₂₀ aliphatic groups, and more preferably C₈-C₁₈ aliphatic groups;
R⁵, R⁶, R⁷ and R⁸ in formula (III) are equal or different and are independently selected from the group consisting of hydrogen, methyl and ethyl, and preferably are hydrogen;
p in formula (III) is an integer number from 0 to 20, preferably an integer number from 1 to 20, more preferably an integer number from 1 to 11, and even more preferably an integer number from 2 to 7; and
R⁹ in formula (IV) is selected from saturated or unsaturated, linear or branched C₅-C₃₀ aliphatic groups, preferably C₆-C₁₈ aliphatic groups, and more preferably C₈-C₁₂ aliphatic groups.

In a preferred embodiment, in the carboxylic acid of formula (III)
R⁴ is a saturated or unsaturated, linear or branched C₈-C₁₈ aliphatic group,
R⁵, R⁶, R⁷ and R⁸ are hydrogen and
p is an integer number from 1 to 11.

In a more preferred embodiment, in the carboxylic acid of formula (III)
R⁴ is a saturated or unsaturated, linear or branched C₈-C₁₈ aliphatic group,
R⁵, R⁶, R⁷ and R⁸ are hydrogen and
p is an integer number from 2 to 7.

In a further preferred embodiment, the carboxylic acid of formula (IV) is iso-nonanoic acid.

Among the carboxylic acids of the formulae (III) and (IV), the carboxylic acids of the formula (III) are preferred.

Preferably, the reaction for the preparation of the catalyst (C) involving calcium hydroxide (A) and carboxylic acid (B) further involves an acid (AC) having a pK_{A} value of 3 or less, preferably 2 or less, more preferably 0 or less, and even more preferably-3 or less.

Preferably, the acid (AC) is selected from the group consisting of acids of sulfur oxides and phosphorus oxides, more preferably from the group consisting of sulfuric acid, sulfurous acid, sulfonic acids (such as methane sulfonic acid), phosphorus acid, phosphorous acid and phosphonic acids (such as methane phosphonic acid), and even more preferably from the group consisting of sulfuric acid, sulfurous acid and methane sulfonic acid.

In a particularly preferred embodiment, the acid (AC) is sulfuric acid.

Preferably, the acid (AC) is used in the reaction for the preparation of the catalyst (C) thus that the molar ratio of the calcium hydroxide (A) to the acid (AC) is from 5:1 to 1:1, more preferably from 3:1 to 1:1, and even more preferably from 2:1 to 1:1.

It is particularly advantageous to prepare the calcium catalyst (C) by first allowing the calcium hydroxide (A) to react with the carboxylic acid (B), preferably in a solvent as described above, after which the reaction mixture is further treated with the acid (AC).

For the reaction by which the calcium catalyst (C) is prepared, any common reactor may be employed, preferably a reactor with an agitating/mixing means, such as, e.g., a magnetic stirrer, a mechanical stirrer, a static mixer, a blender or a batch disperser. Particularly preferably, the mixing of the components is carried out using a batch disperser.

The preparation of the catalyst (C) is preferably carried out under a pressure of from 0.5 to 2 bar, more preferably from 0.8 to 1.5 bar, and even more preferably from 0.9 to 1.2 bar. In a preferred embodiment, the catalyst (C) is prepared under atmospheric pressure. Furthermore, the catalyst (C) is preferably prepared at a temperature of from −30° C. to 100° C., more preferably from 0° C. to 80° C., and even more preferably from 30° C. to 60° C.

The thus prepared calcium catalyst (C) has a content of Ca²⁺ ions that is preferably between 0.5 wt.-% and 5 wt.-%, more preferably from 1 to 4 wt.-% and even more preferably from 2 to 3 wt.-%. Optionally, the catalyst may be rid of volatile components, such as the solvent, water and other volatile byproducts by employing commonly used methods. Preferably, the volatile components are removed in vacuo, e.g. under a pressure below 0.8 bar, preferably below 0.3 bar, and more preferably below 0.1 bar, and/or at elevated temperatures, e.g. 50 to 180° C., preferably 70 to 150° C., and more preferably 80 to 120° C.

In a particularly preferred embodiment, the volatile compounds are removed on a rotary evaporator at a pressure below 0.1 bar and a temperature of from 80° C. to 120° C.

Preferably, the volatile components are removed before the catalyst (C) is used for the preparation of the product mixture of component Z1) of the hand dish-washing composition according to the invention.

The one or more triglycerides of the formula (II) are not particularly limited and may be natural triglycerides or synthetic triglycerides. For the preparation of the one or more ethoxylated glycerol esters of the formula (I), the one or more triglycerides of the formula (II) may be used in a mixture with by-products occurring in nature together with the triglycerides or generated during their preparation, such as by-products containing free hydroxyl groups, e.g. mono- or diglycerides, or in a purified or enriched form, whereby purification or enrichment can e.g. be achieved by employing column chromatography techniques.

Preferably, the one or more triglycerides of formula (II) are selected from the group of vegetable oils and animal fats known in the art including, but not limited to, abyssinian oil, almond oil, apricot kernel oil, avocado oil, babassu oil, beef tallow, borage oil, canola oil, castor oil, cocoa butter, coconut oil, corn oil, cottonseed oil, crisco shortening, emu oil, grapeseed oil, groundnut oil, hazelnut oil, illipe butter, jojoba oil, kukui nut oil, lard, macadamia nut oil, mango butter, neem oil, olive oil, palm oil, palm kernel oil, peach kernel oil, peanut oil, rapeseed oil, rice bran oil, safflower oil, sesame oil, shea butter, soybean oil, sunflower oil, walnut oil, and wheat germ oil.

More preferably, component Z1) of the hand dish-washing composition according to the invention is prepared from triglycerides of the formula (II) selected from the group consisting of coconut oil and rapeseed oil, preferably from coconut oil.

The product mixture from the preparation of ethoxylated glycerol esters of the formula (I) and in particular a product mixture prepared from ethylene oxide and one or more triglycerides of the formula (II) in the presence of a calcium-based catalyst as described above, may comprise, in addition to the one or more ethoxylated glycerol esters of the formula (I), further substances which may be formed in the preparation of the ethoxylated glycerol esters of the formula (I), e.g. substances according to the formula (XV), (XIX) or (XX).

The respective product mixture may be used as component Z1) in the hand dish-washing compositions according to the invention as obtained from the preparation of the one or more ethoxylated glycerol esters of the formula (I) or the one or more ethoxylated glycerol esters of the formula (I) may be purified or enriched after their preparation and before they are used as component Z1) in the hand dish-washing compositions according to the invention, e.g. by employing column chromatography techniques or by employing ion exchange and subsequent filtration techniques.

In a preferred embodiment of the invention, the respective product mixture is used as component Z1) in the hand dish-washing compositions according to the invention as obtained from the preparation of the one or more ethoxylated glycerol esters of the formula (I), in particular when the one or more ethoxylated glycerol esters of the formula (I) have been prepared from ethylene oxide and one or more triglycerides of the formula (II) in the presence of a calcium-based catalyst as described above.

Preferably, the one or more ethoxylated glycerol esters of the formula (I) are present in component Z1) in an amount of 60 wt.-% or more, more preferably 70 wt.-% or more, even more preferably 75 wt.-% or more and particularly preferably 80 wt.-% or more, in each case based on the total weight of component Z1) of the hand dish-washing composition according to the invention.

More preferably, the one or more ethoxylated glycerol esters of the formula (I) are present in component Z1) in an amount of 60 to 100 wt.-%, more preferably 60 to 99.99 wt.-%, even more preferably 70 to 99.9 wt.-%, particularly preferably 75 to 98 wt.-% and extraordinarily preferably 80 to 95 wt.-%, in each case based on the total weight of component Z1) of the hand dish-washing composition according to the invention.

The one or more polyethyleneglycol diesters of the formula (XV) are present in component Z1) of the hand dish-washing composition according to the invention in an amount of preferably 0 to 25 wt.-%, more preferably 0 to 20 wt.-%, even more preferably 0 to 10 wt.-% and particularly preferably 0 to 5 wt.-%, in each case based on the total weight of component Z1) of the hand dish-washing composition according to the invention. This means that component Z1) does not necessarily have to comprise any polyethyleneglycol diesters of the formula (XV). However, the amount of the one or more polyethyleneglycol diesters of the formula (XV) in component Z1) of the hand dish-washing composition according to the invention, if it is not 0%, is preferably from 0.01 to 25 wt.-%, more preferably from 0.1 to 20 wt.-%, even more preferably from 0.5 to 10 wt.-% and particularly preferably from 1 to 5 wt.-%, in each case based on the total weight of component Z1) of the hand dish-washing composition according to the invention.

The total amount of the substances selected from the group consisting of the one or more substances of the formula (XIX) and the one or more substances of the formula (XX) in component Z1) of the hand dish-washing composition according to the invention, if it is not 0%, is preferably from 0.01 to 10 wt.-%, more preferably from 0.1 to 5 wt.-% and even more preferably from 0.1 to 2 wt.-%, in each case based on the total weight of component Z1) of the hand dish-washing composition according to the invention.

The predominant species in component Z1) of the hand dish-washing composition according to the invention is preferably one or more ethoxylated glycerol esters of the formula (I), in which X¹ is —C(═O)—R¹, X² is —C(═O)—R² and X³ is —C(═O)—R³.

The amount of the one or more ethoxylated glycerol esters of the formula (I), in which X¹ is —C(═O)—R¹, X² is —C(═O)—R² and X³ is —C(═O)—R³, in component Z1) of the hand dish-washing compositions according to the invention preferably is from 70 to 98 wt.-%, more preferably from 75 to 95 wt.-% and even more preferably from 85 to 95 wt.-%, based on the total weight of the ethoxylated glycerol esters of the formula (I) contained in component Z1) of the hand dish-washing compositions according to the invention.

The hydroxyl value of component Z1) of the hand dish-washing composition according to the invention, prepared in the presence of the above defined catalyst (C), measured according to DIN EN ISO 4629-2, is preferably less than 10 mg KOH/g above the hydroxyl value of the one or more triglycerides of the formula (II).

Preferably, the hydroxyl value of component Z1) of the hand dish-washing composition according to the invention is smaller than 10 mg KOH/g. Furthermore, the ratio of CH₂OH groups to alkyl-CH₃ groups in component Z1) of the hand dish-washing composition according to the invention is typically below 0.15, preferably below 0.12, more preferably below 0.08, even more preferably below 0.06, measured as the ratio of the integrals of corresponding signals in proton-NMR spectra.

Preferably, the saponification value of component Z1) of the hand dish-washing composition according to the invention is below 220 mg KOH/g and more preferably below 150 mg KOH/g. Preferably, the saponification value of component Z1) of the hand dish-washing composition according to the invention is above 60 mg KOH/g. The saponification values are measured according to DIN EN ISO 3681.

### Surfactant Component Z2)

A surfactant component Z2), comprising one or more anionic surfactants, is desired, as surfactants contribute to the cleaning performance of the hand dish-washing composition according to the invention. Suitable surfactants are selected from the group consisting of an anionic surfactant or a mixture thereof, as well as a cationic surfactant or a mixture thereof, a non-ionic surfactant or a mixture thereof, a zwitterionic surfactant or a mixture thereof, and an amphoteric surfactant or a mixture thereof.

In a preferred embodiment, the hand dish-washing composition according to the invention may comprise from about 2.5 wt % to 80 wt %, preferably from about 5 wt % to about 60 wt %, more preferably from 7 wt % to 45 wt % of the total composition of surfactant (“wt %” or “wt.-%” means percentage by weight).

The composition of the invention comprises at least one anionic surfactant.

In at least one embodiment, the anionic surfactant is selected from the group consisting of (C10-C20)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, a-sulfo fatty acid esters, alkylbenzenesulfonates, alkyl-phenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono-, di-, and triethanolammonium, and analogous alkylammonium salts.

In at least one embodiment, the anionic surfactant is selected from the group consisting of sulfate, a sulfosuccinate, a sulfoacetate, and/or a sulphonate, preferably an alkyl sulfate and/or alkyl ethoxy sulfate, more preferably a combination of an alkyl sulfate and/or an alkyl ethoxy sulfate with a combined ethoxylation degree less than about 5, preferably less than about 3, more preferably less than about 2.

In at least one embodiment, sulfate or sulfonate surfactant is typically present at level of at least 5 wt %, preferably from about 5 wt % to about 40 wt %, and more preferably from 15 wt % to 30 wt %, and even more preferably from about 15 wt % to 25 wt % of the hand dish-washing composition.

In at least one embodiment, the composition comprises a non-ionic surfactant.

In at least one embodiment, the non-ionic surfactant has an HLB (Hydrophilic Lipophilic Balance) of greater than 12. Optionally, the non-ionic surfactant is selected from the group consisting of ethoxylated or ethoxylated/propoxylated fatty alcohols with a fatty chain comprising from 12 to 22 carbon atoms, ethoxylated sterols, such as stearyl- or lauryl alcohol (EO-7), PEG-16 soya sterol or PEG-10 soya sterol, polyoxyethylene polyoxypropylene block polymers (poloxamers), and mixtures thereof.

In at least one embodiment, the non-ionic surfactant is selected from the group consisting of ethoxylated fatty alcohols, fatty acids, fatty acid glycerides or alkylphenols, in particular addition products of from 2 to 30 mol of ethylene oxide and/or 1 to 5 mol of propylene oxide onto C8- to C22-fatty alcohols, onto C12- to C22-fatty acids or onto alkyl phenols having 8 to 15 carbon atoms in the alkyl group, C12- to C22-fatty acid mono- and diesters of addition products of from 1 to 30 mol of ethylene oxide onto glycerol, addition products of from 5 to 60 mol of ethylene oxide onto castor oil or onto hydrogenated castor oil, fatty acid sugar esters, in particular esters of sucrose and one or two C8- to C22-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate, esters of sorbitan and one, two or three C8- to C22-fatty acids and a degree of ethoxylation of from 4 to 20, polyglyceryl fatty acid esters, in particular of one, two or more C8- to C22-fatty acids and polyglycerol having preferably 2 to 20 glyceryl units, alkyl glucosides, alkyl oligoglucosides and alkyl polyglucosides having C8 to C22-alkyl groups, e.g. decylglucoside or laurylglucoside, and mixtures thereof.

In at least one embodiment, the non-ionic surfactant is selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (Pluronics®), fatty acid alkylol amides, (fatty acid amide polyethylene glycols), N alkyl-, N-alkoxypoly-hydroxy-fatty acid amide, sucrose esters, sorbitol esters, polyglycol ethers, and mixtures thereof.

In at least one embodiment, the hand dish-washing composition according to the invention comprises a fatty N-methyl-N-glucamide surfactant. In at least one embodiment, the fatty N-methyl-N-glucamide surfactant conforms to the formula (X):

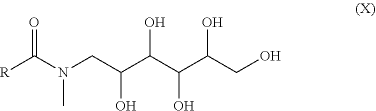

wherein R is a linear or branched alkyl or alkenyl group having from 3 to 30 carbon atoms. In at least one embodiment, R is an alkyl group having from 3 to 30 carbon atoms. In at least one embodiment, R is a saturated aliphatic hydrocarbon group which can be linear or branched and can have from 3 to 20 carbon atoms in the hydrocarbon chain, preferably linear or branched. Branched means that a lower alkyl group such as methyl, ethyl or propyl is present as substituent on a linear alkyl chain. In at least one embodiment, R is selected from the group consisting of 1-propyl, 2-propyl, 1-butyl, 2-butyl, 2-methyl-1-propyl (isobutyl), 2-methyl-2-propyl (tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl and 1-octadecyl. Suitable fatty N-methyl-N-glucamide surfactants are described in WO2013/178700 and EP 0 550 637, which are incorporated herein by reference. In at least one embodiment, the N-methyl-N-glucamide surfactant is selected from those conforming to formula (X), wherein R is C12 alkyl or C14 alkyl. In at least one embodiment, the N-methyl-N-glucamide surfactant is selected from those conforming to formula (X), wherein R is C16 alkyl or C18 alkyl.

In at least one embodiment, the composition comprises from 2 wt % to 10 wt %, more preferably from 2 wt % to 7.5 wt %, even more preferably from 2 wt % to 6 wt % non-ionic surfactant.

In at least one preferred embodiment, the composition further comprises an amphoteric surfactant and/or a zwitterionic surfactant.

In at least one embodiment, the amphoteric surfactants are selected from the group consisting of N—(C12-C18)-alkyl-beta-aminopropionates and N—(C12-C18)-alkyl-beta-iminodipropionates as alkali metal salts and mono-, di-, and trialkylammonium salts; N-acylaminoalkyl-N,N-dimethylacetobetaine, preferably N—(C8-C18)-acylaminopropyl-N,N-dimethylacetobetaine, (C12-C18)-alkyl-dimethyl-sulfopropylbetaine, amphosurfactants based on imidazoline (trade name: Miranol®, Steinapon®), preferably the sodium salt of 1-(beta-carboxymethyloxyethyl)-1-(carboxy-methyl)-2-laurylimidazolinium; amine oxide, e.g., (C12-C18)-alkyl-dimethyl-amine oxide, fatty acid amidoalkyldimethylamine oxide, and mixtures thereof.

In at least one embodiment, the composition comprises a betaine surfactant. Optionally, the betaine surfactant is selected from C8- to C18-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl) alphacarboxyethylbetaine and combinations thereof. Optionally, the betaine surfactant is selected from C8- to C18-sulfobetaines. In at least one embodiment, the betaine surfactant is selected from the group consisting of cocodimethylsulfopropylbetaine, stearyldimethylsulfopropylbetaine, lauryldimethyl-sulfoethylbetaine, laurylbis(2-hydroxyethyl) sulfopropylbetaine, and combinations thereof. Optionally, the betaine surfactant is selected from carboxyl derivatives of imidazole, the C8- to C18-alkyldimethylammonium acetates, the C8- to C18-alkyldimethylcarbonylmethylammonium salts, and the C8- to C18-fatty acid alkylamidobetaines, and mixtures thereof. Optionally, the C8- to C18-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

Suitable zwitterionic surfactant in the preferred embodiment wherein contains both basic and acidic groups which form an inner salt giving both cationic and anionic group is a quaternary ammonium group, although other positively charged groups like phosphonium, imidazolium and sulfonium groups can be used. The typical anionic hydrophilic groups are carboxylate and sulphonate, although other groups like sulfate, phosphonate, and the like can be used. Most preferred amine oxides are coconut dimethyl amine oxide or coconut amido propyl dimethyl amine oxide. Amine oxide may have a linear or mid-branched alkyl moiety. Typical linear amine oxides include water-soluble amine oxide containing one R⁴ C₈₋₁₈ alkyl moiety and 2 R⁵ and R⁸ moieties selected from the group consisting of C₁₋₃ alkyl group and a mixture thereof; and a C₁₋₃ hydroxyalkyl group and a mixture thereof. Preferably amine oxide is characterized by the formula R⁴—N(R⁵)(R⁸)→O wherein R⁴ is C₈₋₁₈ alkyl and R⁵ and R⁸ are selected from the group consisting of a methyl; an ethyl; a propyl; and isopropyl, a 2-hydroxyethyl; a 2-hydroxypropyl; and a 3-hydroxypropyl. The linear amine oxide surfactant, in particular, may include linear C₁₀₋₁₈ alkyl dimethyl amine oxide and a linear C₈-C₁₂ alkoxy ethyl dihydroxy ethyl amine oxide. Preferred amine oxides include linear C₁₀, linear C₁₀-C₁₂ and linear C₁₂-C₁₄ alkyl dimethyl amine oxides.

As used herein “mid-branched” means that the amine oxide has one alkyl moiety having n₁ carbon atoms with one alkyl branch on the alkyl moiety having n₂ carbon atoms. The alkyl branch is located on the alpha carbon from the nitrogen on the alkyl moiety. This type of branching for the amine oxide is also known in the art as an internal amine oxide. The total sum of n₁ and n₂ is from 10 to 24 carbon atoms, preferably from 12 to 20, and even more preferably from 10 to about 16. The number of carbon atoms for the one alkyl moiety (n₁) should be approximately the same number of carbon atoms as the one alkyl branch (n₂) such that the one alkyl moiety and the one alkyl branch are symmetric. As used herein “symmetric” means that |n₁-n₂| is less than or equal to about 5, preferably about 4, most preferably from 0 to 4 carbon atoms in at least 50 wt %, more preferably about 75 wt % to about 100 wt % of the mid-branched amine oxide for use herein. The amine oxide further comprises two moieties, independently selected from C₁₋₃ alkyl;
a C₁₋₃ hydroxyalkyl group, or a polyethylene oxide group containing an average of from about 1 to 3 ethylene oxide groups. Preferably the two moieties are selected from the C₁₋₃ alkyl, more preferably both are selected as a C₁ alkyl.

The zwitterionic and amphoteric surfactant for use in a hand dish-washing composition can be comprised at a level of from about 0.01 wt % to about 20 wt %, preferably from about 0.2 wt % to about 15 wt %, and even more preferably from about 0.5 wt % to about 10 wt % of the hand dish-washing composition.

In one of the preferred embodiments, the hand dish-washing composition can comprise a cationic surfactant present in an effective amount, more preferably from 0.1 wt % to about 20 wt % of the hand dish-washing composition. Suitable cationic surfactant is quaternary ammonium surfactant. Suitable quaternary ammonium surfactant is selected from the group consisting of mono C₆-C₁₆, preferably a C₆-C₁₀ N-alkyl or an alkenyl ammonium surfactant or a mixture thereof, wherein the remaining N positions are substituted by a methyl, a hydroxyethyl or a hydroxypropyl group. Another preferred cationic surfactant is a C₆-C₁₈ alkyl or alkenyl ester of a quaternary ammonium alcohol, such as quaternary chlorine ester. More preferably, the cationic surfactant has a formula (Y):

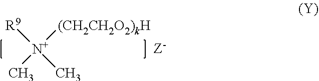

wherein R⁹ of formula (Y) is C₈-C₁₈ hydrocarbyl or mixture thereof, preferably, a C₈-C₁₄ alkyl, more preferably, a C₈, C₁₀ or C₁₂ alkyl; and Z of formula (Y) is an anion, preferably, a chloride or bromide.

### pH Adjustment Component Z3)

### Alkaline Material

The hand dish-washing composition according to the invention has a pH value of from 4.5 to 8.5. Alkaline material may be present to adjust the pH/maintain the pH of the composition according to the present invention. The amount of alkaline material, if present, preferably is from about 0.001 wt % to about 20 wt %, more preferably from about 0.01 wt % to about 10 wt %, and even more preferably from about 0.05 wt % to about 3 wt % of the composition.

Examples of the alkaline material are sodium hydroxide, potassium hydroxide, lithium hydroxide and magnesium oxide, and mixtures thereof. Optionally, the pH adjusting agent may be selected from diethanolamine, triethanolamine, N-methyl ethanolamine, isopropanolamine, diisopropanolamine, triisopropanolamine, glucamine. Preferably, the source of alkalinity is sodium hydroxide or potassium hydroxide, preferably sodium hydroxide.

### Acid

To establish the desired acidic pH value, the hand dish-washing composition according to the invention may comprise an acid. Any acid known to those skilled in the art may be used herein. Typically, the composition herein may comprise up to 20%, preferably from about 0.1% to about 10%, more preferably from 0.1% to about 5%, most preferably from 0.1% to about 3%, by weight of the total composition, of acid, if present.

In at least one embodiment, the composition comprises an acid selected from the group consisting of hydrochloric acid, phosphoric acid, acetic acid, formic acid, sulfuric acid, hydrochloric acid, citric acid, and mixtures thereof. Citric acid is most preferred in that it has high consumer acceptance.

In at least one embodiment, the acidic pH is adjusted with a buffer such as a phosphate buffer, a TRIS buffer or a citric buffer. The buffers may be used alone or in combination with an acid.

### Optional Ingredients Z4)

The hand dish-washing composition according to the invention may comprise a variety of optional ingredients Z4) depending on the technical benefit aimed for.

Suitable optional ingredients for the use herein include an enzyme or a mixture thereof, a perfume or a mixture thereof; an opacifier and/or a pearlizer or mixtures thereof; a dye or mixture thereof; a corrosion inhibitor or mixture thereof; a preservative or mixtures thereof; a disinfectant or a mixture thereof; a chelating agent or a mixture thereof; a hydrotrope or a mixture thereof; a skin protection agent or mixture there of; drying performance modifying agent or mixture thereof; a viscosity modifier such as inorganic salts, in particular sodium chloride, or a mixture thereof.

### Solvent Component Z5)

The hand dish-washing composition according to the invention comprises a solvent component comprising water. In at least one embodiment, the composition comprises a solvent, wherein the solvent comprises water and alcohol. Solvent is useful for providing the hand dish-washing composition according to present invention in liquid form. In at least one embodiment, hand dish-washing composition according to the present invention comprises water from about 5 wt % to about 90 wt %, preferably about 20 wt % to 60 wt % of the composition.

Optionally the composition comprises water-miscible or water-soluble solvents such as lower alkyl alcohols. In at least one embodiment, the composition comprises C1-C5 alkyl monohydric alcohols, preferably C2-C3 alkyl alcohols. The alcohols which may be present are in particular lower monohydric or polyhydric alcohols having 1 to 4 carbon atoms customarily used for cosmetic purposes, such as preferably ethanol and isopropanol.

Optionally, the composition comprises a water-soluble polyhydric alcohol. In at least one embodiment, the water-soluble polyhydric alcohols are polyhydric alcohols having two or more hydroxyl groups in the molecule. In at least one embodiment, the water-soluble polyhydric alcohol is selected from the group consisting of: dihydric alcohols such as ethylene glycol, propylene glycol, trimethylene glycol, 1,2-butylene glycol, 1,3-butylene glycol, 1,4-butylene glycol, tetramethylene glycol, 2,3-butylene glycol, pentamethylene glycol, 2-butene-1,4-diol, hexylene glycol, octylene glycol; trihydric alcohols such as glycerine, trimethylol propane, 1,2,6-hexanetriol and the like; tetrahydric alcohols such as penthaerythritol; pentahydric alcohols such as xylytol, etc.; hexahydric alcohols such as sorbitol, mannitol; polyhydric alcohol polymers such as diethylene glycol, dipropylene glycol, polyethylene glycol, polypropylene glycol, tetraethylene glycol, diglycerine, polyethylene glycol, triglycerine, tetraglycerine, polyglycerine; dihydric alcohol alkyl ethers such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isopropyl ether, ethylene glycol dimethyl ether, ethylene glycol diethyl ether, ethylene glycol dibutyl ether; dihydric alcohol alkyl ethers such as diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether; dihydric alcohol ether esters such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate; glycerine monoalkyl ethers such as xyl alcohol, selachyl alcohol, batyl alcohol; sugar alcohols such as sorbitol, maltitol, maltotriose, mannitol, sucrose, erythritol, glucose, fructose, starch sugar, maltose, xylytose, starch sugar reduced alcohol, glysolid, tetrahydrofurfuryl alcohol, POE tetrahydrofurfuryl alcohol, POP butyl ether, POP POE butyl ether, tripolyoxypropylene glycerine ether, POP glycerine ether, POP glycerine ether phosphoric acid, POP POE pentanerythritol ether, and mixtures thereof.

In a preferred embodiment, the composition comprises a solvent selected from the group consisting of water, glycols, ethanol, and combinations thereof.

In a preferred embodiment, the composition comprises an aqueous, alcoholic or aqueous-alcoholic solvent, and wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, glycerol, or a mixture thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

### Product Format

The composition according to the invention is in liquid form and may be used neat or diluted.

For the hand dish-washing purposes according to the invention, the composition is typically applied neat directly to the surface of table- and/or kitchenware. When applied in a diluted form, the composition is preferably diluted with water in a ratio of between 1:1 to 1:10. The term “diluted” includes the case where the composition of the invention is added to a body of water in which the table- and/or kitchenware is immersed for cleaning.

Only manual dish-washing is considered in the context of the present invention.

The composition may be packaged in the form of any commercially available bottle for storing the liquid.

### Process

In a second aspect, the invention relates to a process of reducing foam during rinse in dish-washing comprising the steps of applying onto table- and/or kitchenware in neat or diluted form a liquid composition according to the invention, cleaning the table- and/or kitchenware with a cleaning implement; and rinsing the table- and/or kitchenware. The term “in diluted form” also includes the case where the composition of the invention is added to a body of water in which the table- and/or kitchenware is immersed for cleaning.

The composition may be applied by any known ways such as by using a scrub, sponge paper, cloth, wipes or any other direct application. The applied composition may be cleaned using a cleaning implement such as a scrub, sponge, paper, cloth or wipes.

The composition according to the invention shows a good water-sheeting effect and is, thus, useful for providing anti-foaming activity upon rinse.

In a further aspect of the invention component Z1 according to the invention is used in a hand dish-washing composition, in particular for providing anti-foaming activity upon rinse and for providing a water-sheeting effect resulting in faster and spotless drying.

A subject matter of the invention is the use of component Z1 according to the invention in a hand dish-washing composition, in particular for providing anti-foaming activity upon rinse and for providing faster and spotless drying, especially of dishware.

The invention will now be illustrated by means of the following non-limiting examples.

### EXAMPLES

### Synthesis Example 1

Methods of Preparation of a Calcium-Based Catalyst (C) with Carboxylic Acid of Formula (III)
a) A mixture of 1047.0 g of a carboxylic acid of formula (III) under the trade name “Emulsogen COL 050” marketed by Clariant Produkte (Deutschland) GmbH, 55.8 g of calcium hydroxide and 360.6 g of propan-2-ol was agitated at ambient temperature for 5 minutes with a batch disperser (Ultra Turrax from IKA Werke GmbH & Co KG). After this, 44.2 g of concentrated sulfuric acid were added over 2 minutes and the mixture was again agitated for 5 minutes with the batch disperser, providing a catalyst with a Ca²⁺ content of 2.00 wt.-% (henceforth “(C-1)”)

Similar results for providing the catalyst with a Ca²⁺ content of approximately 2.00 wt.-% can be obtained by using methane-sulfonic acid or sulfurous acid instead of sulfuric acid.
b) A mixture of 1047.0 g of a carboxylic acid of formula (III) under the trade name “Emulsogen COL 050” marketed by Clariant Produkte (Deutschland) GmbH, 55.8 g of calcium hydroxide and 360.6 g of propan-2-ol was agitated at ambient temperature for 5 minutes with a batch disperser (Ultra Turrax from IKA Werke GmbH & Co KG). After this, 42.9 g of methanesulfonic acid (99 wt.-%) were added over 2 minutes and the mixture was again agitated for 5 minutes with the batch disperser, providing a catalyst with a Ca²⁺ content of 2.00 wt.-% (“(C-3)”).
c) A mixture of 1047.0 g of a carboxylic acid of formula (III) under the trade name “Emulsogen COL 050” marketed by Clariant Produkte (Deutschland) GmbH, 55.8 g of calcium hydroxide and 360.6 g of propan-2-ol was agitated at ambient temperature for 5 minutes with a batch disperser (Ultra Turrax from IKA Werke GmbH & Co KG). After this, 603.7 g of sulfurous acid (6 wt.-%) were added over 2 minutes and the mixture was again agitated for 5 minutes with the batch disperser. The solvent mixture was removed under vacuum, providing a catalyst with a Ca²⁺ content of approximately 2 wt.-% (“(C-4)”).
   Emulsogen COL 050 is a commercial product carboxylic acid (B) comprising, as main component, a carboxylic acid represented by formula (III) wherein R⁴ is oleyl; R⁵, R⁶, R⁷ and R⁸ are hydrogen; and p is 5.

### Synthesis Example 2

### Synthesis of Component Z1): Ethoxylated Coconut Oil (45 EO)

The coconut oil (1 molar equivalent) and the catalyst (C-1)(0.8% by weight, based on the total weight of the mixture of coconut oil and ethylene oxide), were placed into a glass autoclave, which was then flushed with nitrogen by alternatingly applying vacuum and introducing nitrogen (3 cycles). The mixture was dried under aspirator vacuum at 100° C. for 1 hour. The pressure in the autoclave was restored to ambient with nitrogen and heated to 175° C. At this temperature the autoclave was pressurized with nitrogen to a pressure of 0.8 bar above atmospheric pressure, after which pressure-controlled dosage of ethylene oxide (45 molar equivalents) took place up to a maximum pressure of 4.5 bar above atmospheric pressure.

The ethoxylation is carried out in a semi-batch process with automated dosage of ethylene oxide within a given temperature window and up to the specified maximum pressure. The pressure is adjusted according to the increased filling volume of the vessel. After introduction of the intended amount of ethylene oxide and closing the ethylene oxide inlet, the reaction was continued until the pressure became constant. The reactor content was cooled to 90° C. and aspirator vacuum was applied for 30 minutes in order to remove residual ethylene oxide. The temperature was reduced to 80° C. and the final product was transferred into storage vessels and analyzed. The typical batch scale was 400 g to 2000 g. The uptake of the intended amount of ethylene oxide is assured by gravimetry and by determination of the saponification value according to DIN EN ISO 3681.

Analytical data: saponification value=65.0 mg KOH/g; OH-value=4.4 mg KOH/g; content polyethyleneglycol diester represented by the general formula (XV)=3 wt.-%.

Materials Table 1

| | | | | Manufacturer/ |
|---|---|---|---|---|
| Name | | Chemical/Material | Grade | Supplier |
| Genpol LRO | Anionic | SLES 2EO (Sodium | Industrial | Clariant |
| paste | surfactant | Lauryl ether sulfonate) | grade | |
| | | (70% active content) | | |
| Texapon K12 | Anionic | Sodium Lauryl Sulfate | Industrial | BASF |
| | surfactant | | grade | |
| Glucopure | Nonionic | N- Methyl- cocoyl- | Industrial | Clariant |
| Foam | surfactant | glucamine (60% active | grade | |
| | | content) | | |
| Jarfactant 420 | Nonionic | Alkyl Polyglucoside C8- | Industrial | Jarchem |
| UP | surfactant | C16 (37% active | grade | |
| | | content) | | |
| Component | Nonionic | Ethoxylated triglyceride | Industrial | Clariant |
| Z1) | surfactant | oil (coconut oil + | grade | |
| | | 45 EO from Synthesis | | |
| | | example 2) | | |
| Dehyton PK | Amphoteric | CAPB (Cocoamidopropyl | Industrial | BASF |
| 45 | Surfactant | betaine) (37% active | grade | |
| | | content) | | |
| Genaminox | Amphoteric | Lauryldimethylamine | Industrial | Clariant |
| LA | Surfactant | oxid (30% active | grade | |
| | | content) | | |

### Methods

### Preparing the Composition:

The compositions were prepared in batch size of 100 grams each. De-mineralized water and solvents were put in a 250 ml beaker and mixed with anionic surfactants while stirring with a magnetic stirrer at 100 rpm. Needed amount of salt was then added to the mixture. After complete solubilization of the salt, the amphoteric surfactant was added along with the non-ionic surfactant. The mixture was stirred till it was homogenous. Finally, the pH was adjusted to 7.5 by adding Citric acid or sodium hydroxide depending on composition of formulation and stirred for about 2 to 3 minutes. The mixtures were allowed to stand for 24 hours before being examined.

| Composition | A | B | C | D | E | F | G | H |
|---|---|---|---|---|---|---|---|---|
| Genpol LRO | 16 | 16 | 16 | 16 | 16 | 16 | 16 | 16 |
| paste | | | | | | | | |
| Texapon | 10 | 10 | 10 | 10 | 10 | 10 | 10 | 10 |
| K12 | | | | | | | | |
| Glucopure | | | | | | | 4 | 4 |
| Foam | | | | | | | | |
| Component | 0.5 | | 0.5 | | 0.5 | | 0.5 | |
| Z1) | | | | | | | | |
| Jarfactant | | | | | 4 | 4 | | |
| 420 UP | | | | | | | | |
| Dehyton PK | | | 6 | 6 | 2 | 2 | 2 | 2 |
| 45 | | | | | | | | |
| Genaminox | 6 | 6 | | | | | | |
| LA | | | | | | | | |
| Sodium | 1 | 1 | 1 | 1 | 1 | 1 | 1 | 1 |
| Chloride | | | | | | | | |
| Ethanol | 3 | 3 | 3 | 3 | 4 | 4 | 4 | 4 |
| Water | balance | balance | balance | balance | balance | balance | balance | balance |

### Procedure for Measurement of Foam Volume:

6 g/L of the test solution was prepared in 13° dH water. 20 mL of the test solution was taken in a 200 mL graduated glass cylinder. The solution was shook by covering the opening of the cylinder and inverting it 10 times. Then the cylinder was placed on a flat surface of a table for 1 minute for the aqueous layer to separate and it was shook once again to even out the foam level. The volume of foam (excluding aliquote water), in mL was measured and recorded as the initial foam volume. To measure the foam in the first and further rinse cycles, the aliquote water was decanted along the sides of the cylinder retaining foam in the cylinder. 20 ml of fresh 13° dH water was added along the sides of the cylinder and the solution was shook and the foam volume measured as mentioned for initial foam measurement above. The cycles were repeated until no foam is produced anymore.

| | Foam volume per rinse cycle [ml] | | | | | | | | | | |
|---|---|---|---|---|---|---|---|---|---|---|---|
| | Initial | | | | | | | | | | |
| Composition | foam | 1 | 2 | 3 | 4 | 5 | 6 | 7 | 8 | 9 | 10 |
| A | 90 | 100 | 95 | 85 | 35 | 10 | 3 | 0 | | | |
| B | 85 | 100 | 90 | 90 | 80 | 70 | 40 | 20 | 7 | 3 | 0 |
| C | 80 | 95 | 90 | 65 | 30 | 5 | 2 | 0 | | | |
| D | 80 | 90 | 90 | 85 | 75 | 65 | 40 | 15 | 8 | 4 | 0 |
| E | 85 | 90 | 80 | 60 | 20 | 3 | 1 | 0 | | | |
| F | 85 | 95 | 90 | 75 | 50 | 25 | 7 | 2 | 0 | | |
| G | 55 | 60 | 30 | 8 | 3 | 0 | | | | | |
| H | 60 | 60 | 30 | 15 | 8 | 3 | 1 | 0 | | | |

### Procedure for Measurement of Cleaning Performance:

Soiled plates are washed by hand in a 0.5 g/L detergent solution at 45° C. until the foam layer on top of the washing liquor is decomposed. The test result is the number of plates.

Each plate is soiled by 5 g IKW II soil consisting of beef tallow, vegetable fat, margarine, butter, sunflower oil, olive oil, crème fraiche, skimmed milk powder, lard, and dye. The soiled plates are allowed to stand for at least 10 min.

To generate the initial foam, 5 liter of water fall from 1 meter into a dishpan in which 2.5 g of hand dish-washing composition is placed in its middle. Afterwards the soiled plates are washed by using a standardized plastic brush. So many plates are washed until the foam layer in top of the washing liquor is decomposed.

| Composition | Number of plates washed until foam decomposed |
|---|---|
| A | 37 |
| B | 36 |
| C | 30 |
| D | 30 |
| E | 24 |
| F | 24 |
| G | 28 |
| H | 31 |

### Procedure for Measurement of Fast and Spotless Drying:

Five Glasses are washed by hand in a 0.5 g/L detergent solution at 45° C. The test result is the number of spots after drying and time till glass is completely dry. To generate the initial foam, 5 liter of water fall from 1 meter into a dishpan in which 2.5 g of hand dish-washing composition is placed in its middle. Afterwards the glasses are washed by using a standardized glass sink brush. The washed glasses are placed upside down on a drainage board to let them dry at room temperature. The drying time is the time each glass needs to dry completely which is measured by a stopwatch. Afterwards spotting is evaluated by visual observation where + means better than reference and − means worse than reference. Reference in this case means formulation without Component Z1).

Both effects are based on water-sheeting which means that the water runs off faster and without leaving residues.

| Composition | Drying time [min] | Visual evaluation of spotting |
|---|---|---|
| E | 45 | ++ |
| F | 60 | 0 |
| G | 45 | + |
| H | 60 | 0 |

1-19. (canceled)

20. A hand dish-washing composition, which comprises
Z1) a component comprising at least one ethoxylated glycerol ester of the formula (I) 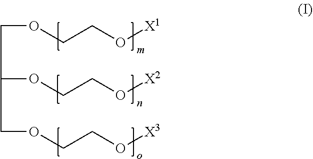
wherein
X¹ is —C(═O)—R¹ or H, X² is —C(═O)—R² or H, X³ is —C(═O)—R³ or H, with the proviso that not all of the groups X¹, X² and X³ are H;
R¹, R² and R³ are equal or different and are independently selected from saturated or unsaturated, linear or branched C7-C24 aliphatic groups; and
m, n and o are equal or different and are each independently an integer number from 1 to 200, with the proviso that the number-average of the sum of m+n+o is greater than 2; and
wherein component Z1) is a product mixture from the preparation of the at least one ethoxylated glycerol ester of the formula (I) in the presence of a calcium-based catalyst;
Z2) a surfactant component, comprising one or more anionic surfactants;
Z3) a pH adjustment component, adjusting the pH value of the composition to from 4.5 to 8.5;
Z4) optionally further ingredients; and
Z5) a solvent component.

21. The hand dish-washing composition according to claim 20, wherein the component Z1) further comprises one or more polyethyleneglycol diesters of the formula (XV) 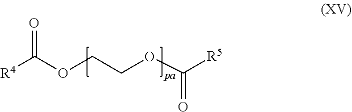
wherein
R⁴ and R⁵ are equal or different and are independently selected from saturated or unsaturated, linear or branched C₇-C₂₄ aliphatic groups; and
pa is an integer number from 1 to 200.

22. The hand dish-washing composition according to claim 20, wherein the at least one ethoxylated glycerol ester of the formula (I) in component Z1) is prepared from 1 to 200 mol ethylene oxide and 1 mol of one or more triglycerides of the formula (II) 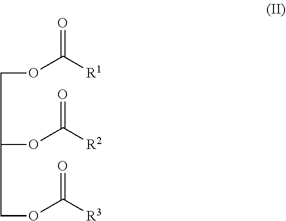
in the presence of a calcium-based catalyst,
wherein R¹, R² and R³ in formulae (II) are equal or different and are independently selected from saturated or unsaturated, linear or branched C₇-C₂₄ aliphatic groups.

23. The hand dish-washing composition according to claim 20, wherein the at least one ethoxylated glycerol ester of the formula (I) in component Z1) is prepared with a calcium-based catalyst (C) obtainable by a reaction involving
(A) calcium hydroxide and
(B) a carboxylic acid comprising 3 to 40 carbon atoms,
wherein the molar ratio of calcium hydroxide (A) to carboxylic acid (B) in the preparation of the catalyst (C) is from 1:1 to 1:5.

24. The hand dish-washing composition according to claim 23, wherein the reaction for the preparation of the catalyst (C) involves a carboxylic acid (B) represented by formula (III) or formula (IV) 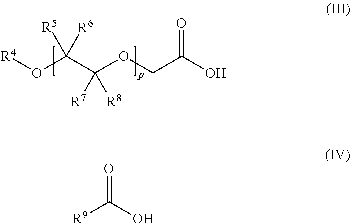
wherein
R⁴ in formula (III) is selected from saturated or unsaturated, linear or branched C₁-C₃₀ aliphatic groups;
R⁵, R⁶, R⁷ and R⁸ in formula (III) are equal or different and are independently selected from the group consisting of hydrogen, methyl, and ethyl;
p in formula (III) is an integer number from 0 to 20; and
R⁹ in formula (IV) is selected from saturated or unsaturated, linear or branched C₅-C₃₀ aliphatic groups.

25. The hand dish-washing composition according to claim 23, wherein the reaction for the preparation of the catalyst (C) involving calcium hydroxide (A) and carboxylic acid (B) further involves an acid (AC) having a pK_{A} value of 3 or less and the molar ratio of (A):(AC) is from 5:1 to 1:1.

26. The hand dish-washing composition according to claim 20, wherein the at least one ethoxylated glycerol ester of the formula (I) in component Z1) is prepared from coconut oil or rapeseed oil.

27. The hand dish-washing composition according to claim 20, wherein the at least one ethoxylated glycerol ester of the formula (I) is present in component Z1) in an amount of 60 wt.-% or more based on the total weight of component Z1) of the hand dish-washing composition.

28. The hand dish-washing composition according to claim 20, wherein the hydroxyl value of component Z1) is smaller than 10 mg KOH/g.

29. The hand dish-washing composition according to claim 20, wherein the composition comprises from about 0.05 to 5% by weight of the hand dish-washing composition of component Z1).

30. The hand dish-washing composition according to claim 20, wherein the surfactant component Z2 comprises at least one anionic surfactant selected from alkyl ether sulphates, linear alkylbenzene sulphonates, alkyl sulphates, and mixtures thereof.

31. The hand dish-washing composition according to claim 20, wherein the surfactant component Z2) further comprises an amphoteric surfactant selected from betaines.

32. The hand dish-washing composition according to claim 20, wherein the surfactant component Z2) further comprises a zwitterionic surfactant and/or a nonionic surfactant selected from fatty N-methyl-N-glucamides, alkyl polyglucosides, and amine oxides.

33. The hand dish-washing composition according to claim 20, wherein the surfactant component Z2) further comprises lauryl dimethyl amine oxide or a fatty N-methyl-N-glucamide surfactant.

34. The hand dish-washing composition according to claim 20, wherein the surfactant component Z2) further comprises a fatty N-methyl-N-glucamide surfactant according to the formula (X): 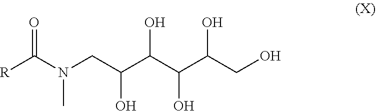
wherein R is a linear or branched alkyl or alkenyl group having from 3 to 30 carbon atoms.

35. The hand dish-washing composition according to claim 20, wherein the composition comprises at least one additional component Z4) selected from the group consisting of enzymes, perfumes, opacifiers and/or a pearlizers, dyes, corrosion inhibitors, preservatives, disinfectants, chelating agents, hydrotropes, skin protection agents, drying performance modifying agents, viscosity modifiers, and mixtures thereof.

36. A process of reducing foam and/or providing a water-sheeting effect resulting in faster and spotless drying during rinse in dishwashing, wherein the process comprises the steps of:
a. applying onto tableware and/or kitchenware in neat or diluted form the liquid composition according to claim 20;
b. cleaning the tableware and/or kitchenware with a cleaning implement; and
c. rinsing the tableware and/or kitchenware.
